(19) United States
(12) Patent Application Publication
(10) Pub. No.: US 2022/0298577 A1
(43) Pub. Date: Sep. 22, 2022

(54) METHYLATION-BASED MODIFIED TUMOR MARKER STAMP-EP8 AND APPLICATION THEREOF

(71) Applicant: SHANGHAI EPIPROBE BIOTECHNOLOGY CO., LTD., Shanghai (CN)

(72) Inventors: Zhenyan LI, Shanghai (CN); Shihua DONG, Shanghai (CN)

(73) Assignee: SHANGHAI EPIPROBE BIOTECHNOLOGY CO., LTD., Shanghai (CN)

(21) Appl. No.: 17/594,792

(22) PCT Filed: Apr. 29, 2020
(86) PCT No.: PCT/CN2020/087885

§ 371 Date: May 20, 2022

(30) Foreign Application Priority Data
Apr. 30, 2019 (CN) 201910363470.7

(51) Int. Cl. C12Q 1/6886

(52) U.S. Cl. CPC C12Q 1/6886; C12Q 2600/154

(57) ABSTRACT

Provided are an isolated polynucleotide and a use thereof, wherein the isolated polynucleotide is the polynucleotide shown in SEQ ID NO:1 or a fragment or complementary nucleic acid thereof at a modified CpG site. Further provided are the use of the polynucleotide for preparing a tumor detection reagent or a kit, a preparation method, a reagent and a use thereof, a kit, and a method for the in-vitro detection of the methylation profile of a sample.

### TECHNICAL FIELD

The disclosure is in the field of markers for diagnosing diseases. More specifically, the disclosure relates to a methylation based tumor marker STAMP, Specific Tumor Aligned Methylation of Pan-cancer, and use thereof.

### BACKGROUND OF DISCLOSURE

Tumors have been considered a genetic disease for decades. Several large-scale systematic sequencings for human have confirmed that the number of somatic mutations in cancer tissues is significantly less than expected. These results suggest that cancer is not a simple genetic disease.

In order to diagnose tumor, many new tumor markers have been discovered and used for clinical diagnosis in recent years. Before 1980, tumor markers were mainly hormones, enzymes, proteins and other cell secretions, such as carcinoembryonic antigen (CEA) and alpha fetoprotein (AFP) used as markers of gastric cancer, liver cancer and other tumors, carbohydrate antigen 125 (CA125) used as a marker of cervical cancer, and prostate specific antigen (PSA) used as a marker of prostate cancer. Although these tumor markers are still used in clinic, their sensitivity and accuracy have been difficult to meet the clinical needs.

More and more evidences show that small changes in epigenetic regulation play an important role in tumors. Epigenetics is a subject that studies that the heritable change of gene function without a change of DNA sequence, which eventually leads to the change of phenotype. Epigenetics mainly includes DNA methylation, histone modification, changes of microRNA level and other biochemical processes. DNA methylation is one of the epigenetic mechanisms, refers to the process of transferring methyl from S-adenosylmethionine (methyl donor) to specific bases under the catalysis of DNA methyltransferase. However, DNA methylation in mammals mainly occurs at the C of 5′-CpG-3′, which results in 5-methylcytosine (5 mC).

Fluid biopsy is a technique for the diagnosis and prediction of tumors using circulating tumor cells or circulating tumor DNA as detection targets. The technology has many shortcomings. First, the sensitivity and specificity are not good enough. The tumor itself is heterogeneous, including a variety of subtypes of cell populations. The proportion of tumor DNA in clinical samples, especially blood samples, is very low. The existing tumor markers are difficult to meet the sensitivity of clinical requirements, and it is easy to cause misdiagnosis. Second, one marker has good effect only for one or a few kinds of tumors. As the DNA sources in blood are very complex, the existing tumor markers cannot solve the complex problems of tumor source and metastasis. Because of these complexities, it is difficult for many DNA methylation tumor markers to have a unified standard in clinical application, which seriously affects the sensitivity and accuracy of the markers.

In the previous study of the inventor, some tumor markers based on methylation modification were found. However, to provide more ways for tumor diagnosis, it is still necessary to find more new tumor markers.

### SUMMARY OF DISCLOSURE

The object of the disclosure is to provide a method for detecting tumor based on abnormal hypermethylation of specific sites in tumor using DNA methylation modification as a tumor marker.

The first aspect of the present disclosure provides an isolated polynucleotide, including: (a) a polynucleotide with a nucleotide sequence as shown in SEQ ID NO: 1; (b) a fragment of the polynucleotide of (a), having at least one (such as 2-191, more specifically 3, 5, 10, 15, 20, 25, 30, 40, 50, 60, 70, 90, 110, 130, 150, 170, 180, 190) modified CpG site; and/or (c) a nucleic acid (such as the polynucleotide with a nucleotide sequence as shown in SEQ ID No: 3) complementary to the polynucleotide or fragment of (a) or (b).

In a preferable embodiment, the modification includes 5-methylation(5mC), 5 -hydroxymethylation(5 hmC), 5 -formylcytosine(5fC) or 5 -carboxylcytosine(5 -caC).

In a preferable embodiment, for (b), the fragment of the polynucleotide contains bases at: positions 204 to 223 (including methylation sites 021 to 024); positions 1 to 478 (including methylation sites 001 to 040) of SEQ ID NO: 1 or 2; positions 513 to 1040 (including methylation sites 041 to 077) of SEQ ID NO: 1 or 2; positions 1082 to 1602 (including methylation sites 078 to 114) of SEQ ID NO: 1 or 2; positions 1621 to 2117 (including methylation sites 115 to 153) of SEQ ID NO: 1 or 2; positions 2160 to 2700 (including methylation sites 154 to 191) of SEQ ID NO: 1 or 2.

The second aspect of the disclosure provides an isolated polynucleotide, which is converted from the polynucleotide, and as compared with the sequence of the first aspect, its cytosine C of the CpG site(s) with modification is unchanged, and the unmodified cytosine is converted into T or U.

In a preferable embodiment, it is converted from the polynucleotide corresponding to the first aspect by bisulfite treatment. In another preferable embodiment, the polynucleotide includes: (d) a polynucleotide with a nucleotide sequence as shown in SEQ ID NO: 2 or 4; (b) a fragment of the polynucleotide of (d), having at least one (such as 2-191, more specifically 3, 5, 10, 15, 20, 25, 30, 40, 50, 60, 70, 90, 110, 130, 150, 170, 180, 190) CpG site with modification.

In a preferable embodiment, for (e), the fragment of the polynucleotide contains bases at: positions 204 to 223 (including methylation sites 021 to 024); positions 1 to 478 (including methylation sites 001 to 040) of SEQ ID NO: 1 or 2; positions 513 to 1040 (including methylation sites 041 to 077) of SEQ ID NO: 1 or 2; positions 1082 to 1602 (including methylation sites 078 to 114) of SEQ ID NO: 1 or 2; positions 1621 to 2117 (including methylation sites 115 to 153) of SEQ ID NO: 1 or 2; positions 2160 to 2700 (including methylation sites 154 to 191) of SEQ ID NO: 1 or 2; positions 2478 to 2497 (corresponding to methylation sites 021 to 024); positions 2223 to 2700 (corresponding to methylation sites 001 to 040) of SEQ ID NO: 4 or 3; positions 1661 to 2188 (corresponding to methylation sites 041 to 077) of SEQ ID NO: 4 or 3; positions 1099 to 1619 (corresponding to methylation sites 078 to 114) of SEQ ID NO: 4 or 3; positions 584 to 1080 (corresponding to methylation sites 115 to 153) of SEQ ID NO: 4 or 3; positions 1 to 541 (corresponding to methylation sites 154 to 191) of SEQ ID NO: 4 or 3.

The third aspect of the disclosure provides a use of the polynucleotide described in the first or second aspect in manufacture of a tumor detection agent or kit.

In a preferable embodiment, the tumors include (but are not limited to): hematologic cancers such as leukemia, lymphoma, multiple myeloma; gynecological and reproductive system tumors such as breast cancer, ovarian cancer, cervical cancer, vulvar cancer, testicular cancer, prostate cancer, penile cancer; digestive system tumors such as esophageal cancer (cancer of the esophagus), gastric cancer, colorectal cancer, liver cancer, pancreatic cancer, bile duct and gallbladder cancer; respiratory system tumors such as lung cancer, pleuroma; nervous system tumors such as glioma, neuroblastoma, meningioma; head and neck tumors such as oral cancer, tongue cancer, laryngeal cancer, nasopharyngeal cancer; urinary system tumors such as kidney cancer, bladder cancer, skin and other systems tumors such as skin cancer, melanoma, osteosarcoma, liposarcoma, thyroid cancer.

In another preferable embodiment, samples of the tumor include but are not limited to: tissue samples, paraffin embedded samples, blood samples, pleural effusion samples, and alveolar lavage fluid samples, ascites and lavage fluid samples, bile samples, stool samples, urine samples, saliva samples, sputum samples, cerebrospinal fluid samples, cell smear samples, cervical scraping or brushing samples, tissue and cell biopsy samples.

The fourth aspect of the disclosure provides a method of preparing a tumor detection agent, including: providing the polynucleotide described in the first or second aspect, designing a detection agent for specifically detecting the modification on CPG site(s) of a target sequence which is the full length or fragment of the polynucleotide; wherein, the target sequence has at least one (such as 2-191, more specifically, 3, 5, 10, 15, 20, 25, 30, 40, 50, 60, 70, 90, 110, 130, 150, 170, 180, 190) modified CpG site; preferably, the detection agent includes (but is not limited to) primers or probes.

The fifth aspect of the disclosure provides an agent or a combination of agents which specifically detect the modification on CPG site(s) of a target sequence, which is the full length or fragment of any of the polynucleotides described in the first or second aspect and has at least one (such as 2-191, more specifically, 3, 5, 10, 15, 20, 25, 30, 40, 50, 60, 70, 90, 110, 130, 150, 170, 180, 190) modified CpG site.

In a preferable embodiment, the agent or combination of agents is for a gene sequence containing the target sequence (designed based on the gene sequence), and the gene sequence includes gene Panels or gene groups.

In another preferable embodiment, the detection agent comprises: primers or probes.

In another preferable embodiment, the primers are: the primers shown in SEQ ID NO: 3 and 4; the primers shown in SEQ ID NO: 7 and 8; the primers shown in SEQ ID NO: 9 and 10; the primers shown in SEQ ID NO: 11 and 12; the primers shown in SEQ ID NO: 13 and 14; or the primers shown in SEQ ID NO: 15 and 16.

In the sixth aspect of the disclosure, a use of the agent or combination of agents described in the fifth aspect of the disclosure in the manufacture of a kit for detecting tumors is provided; preferably, the tumors include (but are not limited to): hematologic cancers such as leukemia, lymphoma, multiple myeloma; gynecological and reproductive system tumors such as breast cancer, ovarian cancer, cervical cancer, vulvar cancer, testicular cancer, prostate cancer, penile cancer; digestive system tumors such as esophageal cancer, gastric cancer, colorectal cancer, liver cancer, pancreatic cancer, bile duct and gallbladder cancer; respiratory system tumors such as lung cancer, pleuroma; nervous system tumors such as glioma, neuroblastoma, meningioma; head and neck tumors such as oral cancer, tongue cancer, laryngeal cancer, nasopharyngeal cancer; urinary system tumors such as kidney cancer, bladder cancer, skin and other systems tumors such as skin cancer, melanoma, osteosarcoma, liposarcoma, thyroid cancer.

The seventh aspect of the present disclosure provides a detection kit, comprising container(s) and the agent or combination of agents described above in the container(s); preferably, each agent is located in an independent container.

In a preferable embodiment, the kit also includes: bisulfite, DNA purification agent, DNA extraction agent, PCR amplification agent and/or instruction for use (indicating operation steps of the detection and a result judgment standard).

In the eighth aspect of the disclosure, a method for detecting the methylation profile of a sample in vitro is provided, including: (i) providing the sample and extracting the nucleic acid; (ii) detecting the modification on CPG site(s) of a target sequence in the nucleic acid of (i), wherein the target sequence is the polynucleotide described in the first aspect or the polynucleotide converted therefrom as described in the second aspect.

In a preferable embodiment, in step (3), the analysis methods include pyrosequencing, bisulfite conversion sequencing, method using methylation chip, qPCR, digital PCR, second generation sequencing, third generation sequencing, whole genome methylation sequencing, DNA enrichment detection, simplified bisulfite sequencing technology, HPLC, MassArray, methylation specific PCR (MSP), or their combination, as well as in vitro detection and in vivo tracer detection for the combined gene group of partial or all of the methylation sites in the sequence shown in SEQ ID NO: 1. In addition, other methylation detection methods and newly developed methylation detection methods in the future can be applied to the disclosure.

In another preferable embodiment, step (ii) includes: (1) treating the product of (i) to convert the unmodified cytosine into uracil; preferably, the modification includes 5-methylation, 5-hydroxymethylation, 5-formylcytosine or 5-carboxylcytosine; preferably, treating the nucleic acid of step (i) with bisulfite; and (2) analyzing the modification of the target sequence in the nucleic acid treated by (1).

In another preferable embodiment, the abnormal methylation profile is the high level of methylation of C in CPG(s) of the polynucleotide.

In another preferable embodiment, the methylation profile detecting method is not for the purpose of directly obtaining the diagnosis result of a disease, or is not a diagnostic method.

The ninth aspect of the disclosure provides a tumor diagnosis kit, including primer pair(s) designed based on the sequence described in the first or second aspect of the disclosure, and gene Panels or gene group containing the sequence, to obtain the characteristics of normal cells and tumor cells through DNA methylation detection.

Other aspects of the disclosure will be apparent to those skilled in the art based on the disclosure herein.

### BRIEF DESCRIPTION OF DRAWINGS

FIG. 1, BSP assay for methylation levels of the methylation sites 001 to 040 in SEQ ID NO:1 region in lung cancer cells and normal lung cells. Dark squares indicate “methylated”.

FIG. 2, BSP assay for methylation levels of the methylation sites 041 to 077 in SEQ ID NO:1 region in lung cancer cells and normal lung cells. Dark squares indicate “methylated”.

FIG. 3, BSP assay for methylation levels of the methylation sites 078 to 114 in SEQ ID NO:1 region in lung cancer cells and normal lung cells. Dark squares indicate “methylated”.

FIG. 4, BSP assay for methylation levels of the methylation sites 115 to 153 in SEQ ID NO:1 region in lung cancer cells and normal lung cells. Dark squares indicate “methylated”.

FIG. 5, BSP assay for methylation levels of the methylation sites 154 to 191 in SEQ ID NO:1 region in lung cancer cells and normal lung cells. Dark squares indicate “methylated”.

FIG. 6, comparison of methylation values of STAMP-EP8 between non-cancer tissues and cancer tissues for clinical samples of leukemia.

FIG. 7, comparison of methylation values of STAMP-EP8 between paracancerous samples and cancer tissue samples for clinical samples of breast cancer.

FIG. 8, comparison of methylation values of STAMP-EP8 between paracancerous samples and cancer tissue samples for clinical samples of colorectal cancer.

FIG. 9, comparison of methylation values of STAMP-EP8 between paracancerous samples and cancer tissue samples for clinical samples of esophageal cancer.

FIG. 10, comparison of methylation values of STAMP-EP8 between paracancerous samples and cancer tissue samples for clinical samples of liver cancer.

FIG. 11, comparison of methylation values of STAMP-EP8 between paracancerous samples and cancer tissue samples for clinical samples of lung cancer.

FIG. 12, comparison of methylation values of STAMP-EP8 between pancreatic samples and cancer tissue samples for clinical samples of pancreas cancer.

### DETAILED DESCRIPTION

The inventor is committed to the research of tumor markers. After extensive research and screening, the inventor provides a universal DNA methylation tumor marker, STAMP (Specific Tumor Aligned Methylation of Pan-cancer). In normal tissues, STAMP was hypomethylated, while in tumor tissues, it was hypermethylated. It can be used for clinical tumor detection and as the basis of designing tumor diagnostic agents.

### Terms

As used herein, “isolated” refers to a material separated from its original environment (if the material is a natural material, the original environment is the natural environment). For example, in living cells, polynucleotides and polypeptides in their natural state are not isolated or purified, but the same polynucleotides or polypeptides will be isolated ones if they are separated from other substances existed in the natural state.

As used herein, “sample” includes substances suitable for DNA methylation detection obtained from any individual or isolated tissue, cell or body fluid (such as plasma). For example, the samples include but are not limited to: tissue samples, paraffin embedded samples, blood samples, pleural effusion samples, and alveolar lavage fluid samples, ascites and lavage fluid samples, bile samples, stool samples, urine samples, saliva samples, cerebrospinal fluid samples, cell smear samples, cervical scraping or brushing samples, tissue and cell biopsy samples.

As used herein, “hypermethylation” refers to high level of methylation, hydroxymethylation, formylcytosine or carboxylcytosine of CpG in a gene sequence. For example, in the case of methylation specific PCR (MSP), if the PCR reaction with methylation specific primers has positive PCR results, the DNA (gene) region of interest is in hypermethylation state. For another example, in the case of real-time quantitative methylation specific PCR, hypermethylation can be determined based on statistic difference of the methylation status value as compared with the control sample.

### Gene Marker

In order to find a useful target for tumor diagnosis, the inventor has identified the target of STAMP-EP8 after extensive and in-depth research. The methylation status of the sequence of STAMP-EP8 gene is significantly different between tumor tissues and non-tumor tissues. If the abnormal hypermethylation with a nucleotide sequence as of STAMP-EP8 gene of a subject is detected, the subject can be identified as having a high-risk of tumor. Moreover, the significant difference of STAMP-EP8 between tumor and non-tumor tissues exists in different kinds of tumors, including solid tumors and non-solid tumors.

Therefore, the disclosure provides an isolated polynucleotide, comprising the nucleotide sequence shown in the sequence of SEQ ID NO: 1 or SEQ ID NO: 3 (the reverse complementary sequence of SEQ ID NO: 1). For tumor cells, the polynucleotide contains 5-methylcytosine (5mC) at C positions of many 5′-CpG-3′. The disclosure also comprises a fragment of the polynucleotide with a nucleotide sequence as shown in SEQ ID NO: 1 or 3, having at least one (such as 2-191, more specifically 3, 5, 10, 15, 20, 25, 30, 40, 50, 60, 70, 90, 110, 130, 150, 170, 180, 190) methylated CpG site. The above polynucleotides or fragments can also be used in the design of detection agents or detection kits.

In some specific embodiments of the disclosure, the fragment of the polynucleotide is, for example, a fragment containing the residues 281-309 of SEQ ID NO: 1 (containing CpG sites 017-020). Antisense strands of the above fragments are suitable for use in the disclosure. These fragments are merely examples of preferable embodiments of the present disclosure. Based on the information provided by the present disclosure, other fragments can also be selected.

In addition, gene Panels or gene groups containing the sequence shown in the SEQ ID NO: 1 or SEQ ID NO: 2 or a fragment thereof are also encompassed by the disclosure. For the gene Panel or gene group, the characteristics of normal cells and tumor cells can also be identified through DNA methylation detection.

The above polynucleotides can be used as the key regions for analysis of the methylation status in the genome. Their methylation status can be analyzed by various technologies known in the art. Any technique that can be used to analyze the methylation state can be applied to the present disclosure.

When treated with bisulfite, un-methylated cytosine(s) of the above polynucleotides will be converted into uracil, while methylated cytosine(s) remained unchanged.

Therefore, the disclosure also provides the polynucleotides obtained from the above polynucleotides (including the complementary chain (antisense chain)) after being treated with bisulfite, including the polynucleotides with a nucleotide sequence as shown in SEQ ID NO: 2 or SEQ ID NO: 4. These polynucleotides can also be used in the design of detection agents or detection kits.

The disclosure also comprises a fragment of the polynucleotides obtained from the above polynucleotides or the antisense strand thereof after being treated with bisulfite, wherein the fragment contains at least one (such as 2-191, more specifically 3, 5, 10, 15, 20, 25, 30, 40, 50, 60, 70, 90, 110, 130, 150, 170, 180, 190) methylated CpG site. The No. of each CpG site in the antisense strand corresponding to the number of the sense strand is readily obtained according to the content described by the present disclosure.

### Detection Agents and Kits

Based on the new discovery of the disclosure, a detection agent designed based on said polynucleotide(s) is also provided for detecting the methylation profile of polynucleotide(s) in the sample in vitro. The detection methods and agents known in the art for determining the sequence, variation and methylation of the genome can be applied in the disclosure.

Therefore, the disclosure provides a method of preparing a tumor detection agent, including: providing the polynucleotide, designing a detection agent for specifically detecting a target sequence which is the full length or fragment of the polynucleotide; wherein, the target sequence has at least one methylated CpG site.

The detection agent herein includes but is not limited to: primers or probes, etc.

For example, the agent is primer pair(s). Based on the sequence of the polynucleotide, those skilled in the art know how to design primer(s). The two primers are on each side of the specific sequence of the target gene to be amplified (including CpG sequence, for the gene region originally methylated, the primer is complementary with CpG, and for the gene region originally un-methylated, the primer is complementary with TpG). It should be understood that based on the new discovery of the disclosure, those skilled in the art can design a variety of primers or probes or other types of detection agents for CpG sites at different positions on the target sequence or their combinations. These primers or probes or other types of detection agents should be included in the technical solution of the disclosure. In a preferable example of the disclosure, the primers are: the primers shown in SEQ ID NO: 5 and 6, which could be used to amplify a product containing CpG sites 001-040 of SEQ ID NO: 1; or, the primers shown in SEQ ID NO: 7 and 8, which could be used to amplify a product containing CpG sites 041-077 of SEQ ID NO: 1; the primers shown in

SEQ ID NO: 9 and 10, which could be used to amplify a product containing CpG sites 078-114 of SEQ ID NO: 1; the primers shown in SEQ ID NO: 11 and 12, which could be used to amplify a product containing CpG sites 115-153 of SEQ ID NO: 1; the primers shown in SEQ ID NO: 13 and 14, which could be used to amplify a product containing CpG sites 154-191 of SEQ ID NO: 1; the primers shown in SEQ ID NO: 15 and 16, which could be used to amplify a product containing CpG sites 021-024 of SEQ ID NO: 1.

The agent can also be a combination of agents (primer combination), including more than one set of primers, so that the multiple polynucleotides can be amplified respectively.

The disclosure also provides a kit for detecting the methylation profile of polynucleotide in a sample in vitro, which comprises container(s) and the above primer pair(s) in the container(s).

In addition, the kit can also include various reagents required for DNA extraction, DNA purification, PCR amplification, etc.

In addition, the kit can also include an instruction for use, which indicates operation steps of the detection and a result judgment standard, for the application of those skilled in the art.

### Detection Method

The methylation profile of a polynucleotide can be determined by any technique in the art (such as methylation specific PCR (MSP) or real-time quantitative methylation specific PCR, Methylight), or other techniques that are still developing and will be developed.

Quantitative methylation specific PCR (QMSP) can also be used to detect methylation level. It is a continuous optical monitoring method based on fluorescent PCR, which is more sensitive than MSP. It has high throughput and avoids electrophoresis based result analysis.

Other available technologies include conventional methods in the art such as pyrosequencing, bisulfite converstion sequencing, qPCR, second generation sequencing, whole genome methylation sequencing, DNA enrichment detection, simplified bisulfite sequencing or HPLC, and combined gene group detection. It should be understood that, on the basis of the new disclosure herein, these well-known technologies and some technologies to be developed in the art can be applied to the present disclosure.

As a preferable embodiment of the disclosure, a method of detecting the methylation profile of a polynucleotide in a sample in vitro is also provided. The method is based on the follow principle: the un-methylated cytosine can be converted into uracil by bisulfite, which can be transformed into thymine in the subsequent PCR amplification process, while the methylated cytosine remains unchanged; therefore, after the polynucleotide is treated by bisulfite, the methylated site presents a polynucleotide polymorphism (SNP) similar to C/T. Based on the above principle, methylated and un-methylated cytosine can be distinguished by identifying the methylation profile of a polynucleotide in the sample.

The method of the disclosure includes: (a) providing samples and extracting genomic DNA; (b) treating the genomic DNA of step (a) with bisulfite, so as to convert the un-methylated cytosine in the genomic DNA into uracil; (c) analyzing whether the genomic DNA treated in step (b) contains an abnormal methylation profile.

The method of the disclosure can be used for: (i) analyzing whether a subject has tumor by detecting the sample of the subject; (ii) identifying a population having high-risk of tumor. The method needs not to be aimed at obtaining direct diagnosis results.

In a preferable embodiment of the disclosure, DNA methylation is detected by PCR amplification and pyrosequencing. It should be understood by those in the art that DNA methylation detection is not limited to these methods, and any other DNA methylation detection method can be used. The primers used in PCR amplification are not limited to those provided in Examples.

Because of bisulfite treatment, in which un-methylated cytosine in genomic DNA are converted into uracil and then transformed into thymine in the subsequent PCR process, the sequence complexity of the genome will be reduced, and it will be more difficult to amplify specific target fragments by PCR. Therefore, in order to improve amplification efficiency and specificity, nested PCR may be preferable, wherein two sets of primers (outer primers and inner primers) are used in two successive runs of PCR, and the product from the first run undergoes a second run with the second set of primers. However, it should be understood that the detection methods suitable for the present disclosure are not limited thereto.

After the research and verification on clinical samples, the method of the disclosure provides very high accuracy in the clinical diagnosis of tumors. The disclosure can be applied to the fields of tumor auxiliary diagnosis, efficacy evaluation, prognosis monitoring, etc., thus has a high clinical value.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press, 2002) or followed the manufacturer's recommendation.

### EXAMPLE 1

### Nucleic Acid Sequence for STAMP-EP8 Detection

Based on research and screening, a tumor marker STAMP-EP8 is provided and its sequence is as follows: SEQ ID NO: 1 (chr8:23562476-23565175, Human/hg19), in which the underlined bases are methylated CDG sites. and each number below the underline indicates the site number.

```
CGGATCGCTACCGCGGCCTTGGGCTCAGTTCCTGGAGCCGGCCTCGGGTCCGGCCTCGGGTC
1 2 3 4 5 6 7 8
CCTCCTCGGCGGCGTCCACTGCTGCCGCAGAAGAGACGCTCCCAGGATTTGGTTTCCAGTGG
            9 10 11 12 13
GAGAAGGAAGGCGCCGCAAATACCATCGGCTGCGAGGCGACTGTGGTGGTCGAAGGAACG
                      14 15 16 17 18 19 20

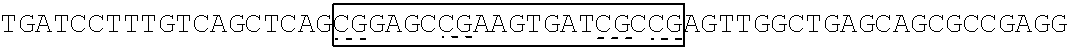

                                      21 22 23 24 25 26
GAGTTTAGAATCCGCTGCAGAAAACCTACCAGCTTCTTCGCGGACACAGCAGGTAATGTTCT
                        27 28 29
TAGGCTTCTGGAGTTAGTGAGTAGCCGGCCTGCAGGCCGGTAAATAACTCCGATCTAGCCC
                                                  30 31 32
CTTGGTTTATTTCTTTCGTTCGCGCCTTGAATGGAGGCGCCATGGCACAGTCTGGCAGAGGA
                                33 34 35 36
GGCCTGCCCTGGCGGGCGCTACTCAGGGCCCCTGCGTTGAGAATGCCGAGTGAGGTGCTGA
                        37 38 39 40
GGTTTGGAGAGAAAAAGGTGGCGTTTTCATCCCTTGGAATATCGGGCCTCTCCAGCTGAAG
                                          41 42
CCTCCGGATCGACACAACACAACTCCCGGATCGACGCAACACAACTCCCCGCCAGCCCCTT
        43 44 45 46 47 48
CCCTTCCAGCGGGCTGGACACAGGTGCGTGGGCCTCGCAGCCCGGCGCGCACGGTTGCGCC
                  49 50 51 52 53 54 55 56
TTTGGATACCTGCGACAGGACTCGGCCAGAACCGTGTCCCAGGGCAGAGAAAACCCTGGCA
                        57 58 59
ACCTTTAACGTTCTCCCTGACGCGGAGAATCCTGCGCCAACGGGGCCTTCCACTTCGGGAGA
                60 61 62 63 64 65
CTCAATCGCCACCCGGTCCCCAACTTTCGTCTTCCAGAAGAAAAATCCATGAGGAGGAATG
            66 67 68
GGGGGGTCCCCCGACCAGACCAGCAAGAAAGGGCCCAGTTAGGAGTGACCTCAGCGCCA
                      69 70
CGAGGGTTCCACTGCTTTGATGGCCACACGCTTTTGCAAACGGTTTGGGGGTGGAGAGACA
71 72 73
CCAGGTGATGGTAGGGAAGGGCGGTGGTGACTGGCACGGCTAAGACACTGCGGAGGGTTT
                                          74 75 76
CGCTGTGGAGATGAGAAGGTGGAGGTTCTGGGCACTGTGGCTGCGAGTGTGAGCGATGCG
77 78 79 80
TTTGTGGGAATCAGAGGAGCGGATTTGGGAAAAGTGAATCCTTGAGATTCAGGGCCTTACT
                                      81
TTCGTTAGGGGTGTGTGAAGCACACTGGGTGTCAGGAGAGGATGGGAATGGGATTCGAGAG
    82 83
GCCTTTTTTTGGACTCCTCGAGAGAAAATGGAGAGAGTCTCGAACCCAGGAGATAGGAGG
                                    84 85
CGTATTTTCCCCATGCACCCATGGATCACGCCCCCGCCCCCACATTCCCCCCGTAGGAGGCA
86 87 88 89
AGACCTGAGCGCTTACTCACGTGGCTCCCCCATCCGTTCCGCGTCCATCTCCAAGACTGCCT
                  90 91 92 93 94
CACAGGGACCCCCAGGAGGCTCCGAACCATCCAGCTTTCTGTCACCGCCGCCGCCACCAG
                                            95 96 97 98
CGTTGTGAACCTCTGACCCTCGCGGCTCTGCGTCCATTCTCAGGTACTGAAAGTTTTCCGGG
99 100 101 102 103
CTCTTCCGCACCCGCGGATGTGGCGAAGCCGCGGGGCAGCTCCGCTCGCGCTCCAGTCGCA
            104 105 106 107 108 109 110 111 112 113
GGATGTCCTTGACCGAGAAGGGGGTGGAGGTGACGGGGCTCAGCAGCATCCCGAAGGCGG
                          114 115 116 117
ATGGGGCGGGGCCGAGGAGGTCCGGGTGAGGAGCGGCACCCTGAACTTCCCGTCTTGTCGC
            118 119 120 121 122 123
TGCAGGCCCCGCAGACAGACCCAAGCTCTGGGACAGACGCCCAGCGTCCCAGACAGCGCCT
                  124 125 126 127
TCCTCTGGGCCATGCTGGTAGGCCCGGGTCCAGGGCCGGGTGACGAGACCGTAGCCCCCCA
                                                128 129 130 131
TTGGTTCTCGCAGAAACCACGTGCTAGCTCTGCACCTTCCTCCCCCAGCGCTTTCTTCCCGCG
                132 133 134 135 136
CCCGTCGAATCCTCTCCAGTCCCCAGTCGCCTTCCTGGGAGGTTTGGTTCGTATTGACGGTT
    137 138 139 140 141
CTCAAAGCAAAGAAAACGTGGAGAGACAGTGCTGCGCGCGGCTTGGATCTCCGTCAATACG
                                142 143 144 145 146 147
GTACTAGCGGGAGCTTCCGAGGTCAACCTAGGGTCCACGGGGACCCTCCTAGTGGACATG
              148 149 150
CGGGGATTGGAGGTGGAGTCCTTGCTGTGCAACCCGATCCGGTCCATCCAGCCTGGTCTTAG
151 152 153
CTCAACTCTGCATTCTTATGGCGGAGGAAAGCTAAGATAGAGACAGCTGGGACCCGCGGAA
                                          154 155 156
TTTGAGGCGCGCGCTGGGATCTTACGTGCGCAGAGGCCGGACCGCAGAGTAGAAGTGAGTC
            157 158 159 160 161 162 163
TTGACCCACCAGGGCCTATATTGAGATGGAAACCTCTCTCTTTGAACAGAATCCAGGTCTG
CGGAGTCCCGACCCGCCACTTCCTAGGAGCAGCTTCTCAGCCCCAGGACCGAGGAATGGGT
164 165 166 167
GTGGGGGCTACATGTGGGGAGCCCTCGACCTCTTAAAGGGCTCCGCGAATGGTGGGGGAGA
                                                  168 169 170
CTGGAAGGCAATCTCTGCGGCCCGCGCAAAGTGGTCAGAGGCGGGAGTGGGGGTGGGGGG
                                  171 172 173 174
GGGTGGGCTTCCGTCGCCCTTGGCGTCCCCACGTTCTTTTTCTTCCTCTCCTTTATCCTCCTTA
                      175 176 177 178
AAGCGGATTGCCTGGAGCGTGCCAGCTTGTGTGCGCGTATAAATGCGGCGCCCGCCGAAGG
      179 180 181 182 183 184 185 186
GCTGGGAAGATTTGACTCGCATTAGATCTCCAGAAAAGGCCCTTGGTTACGGCGGGCACCG
                                  187 188 189 190
AGTAAATGGCG
                  191
```

The bisulfate treated sequence of SEQ ID NO: 1 is shown in SEQ ID NO: 2 (Y represents C or U) as follows:

```
YGGATYGUTAUYGYGGUUTTGGGUTUAGTTUUTGGAGUYGGUUTYGGGTUYGGUUTYGGG
1 2 3 4 5 6 7 8
TUUUTUUTYGGYGGYGTUUAUTGUTGUYGUAGAAGAGAYGUTUUUAGGATTTGGTTTUUA
                  9 10 11 12 13
GTGGGAGAAGGAAGGYGUYGUAAATAUUATYGGUTGYGAGGYGAUTGTGGTGGTYGAAG
                              14 15 16 17 18 19

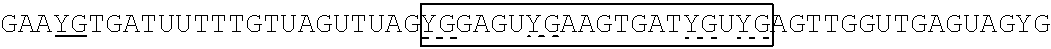

      20 21 22 23 24 25
UYGAGGGAGTTTAGAATUYGUTGUAGAAAAUUTAUUAGUTTUTTYGYGGAUAUAGUAGGT
  26 27 28 29
AATGTTUTTAGGUTTUTGGAGTTAGTGAGTAGUYGGUUTGUAGGUYGGTAAATAAUTUYG
                                                                  30 31 32
ATUTAGUUUUTTGGTTTATTTUTTTYGTTYGYGUUTTGAATGGAGGYGUUATGGUAUAGTU
                                                  33 34 35 36
TGGUAGAGGAGGUUTGUUUTGGYGGGYGUTAUTUAGGGUUUUTGYGTTGAGAATGUYGA
                                            37 38 39 40
GTGAGGTGUTGAGGTTTGGAGAGAAAAAGGTGGYGTTTTUATUUUTTGGAATATYGGGUUT
                                                                  41 42
UTUUAGUTGAAGUUTUYGGATYGAUAUAAUAUAAUTUUYGGATYGAYGUAAUAUAAUTU
                                43 44 45 46 47
UUYGUUAGUUUUTTUUUTTUUAGYGGGUTGGAUAUAGGTGYGTGGGUUTYGUAGUUYGG
    48 49 50 51 52
YGYGUAYGGTTGYGUUTTTGGATAUUTGYGAUAGGAUTYGGUUAGAAUYGTGTUUUAGGG
53 54 55 56 57 58 59
UAGAGAAAAUUUTGGUAAUUTTTAAYGTTUTUUUTGAYGYGGAGAATUUTGYGUUAAYGG
                                                  60 61 62 63 64
GGUUTTUUAUTTYGGGAGAUTUAATYGUUAUUYGGTUUUUAAUTTTYGTUTTUUAGAAGA
                        65 66 67 68
AAAATUUATGAGGAGGAATGGGGGGGTUUUUYGAUUAGAUUAGUAAGAAAGGGUUUAGT
                                                              69
TAGGAGTGAUUTUAGYGUUAYGAGGGTTUUAUTGUTTTGATGGUUAUAYGUTTTTGUAAA
                              70 71 72
YGGTTTGGGGGTGGAGAGAUAUUAGGTGATGGTAGGGAAGGGYGGTGGTGAUTGGUAYGG
73 74 75
UTAAGAUAUTGYGGAGGGTTTYGUTGTGGAGATGAGAAGGTGGAGGTTUTGGGUAUTGTG
                      76 77
GUTGYGAGTGTGAGYGATGYGTTTGTGGGAATUAGAGGAGYGGATTTGGGAAAAGTGAAT
        78 79 80 81
UUTTGAGATTUAGGGUUTTAUTTTYGTTAGGGGTGTGTGAAGUAUAUTGGGTGTUAGGAGA
                                                82
GGATGGGAATGGGATTYGAGAGGUUTTTTTTTGGAUTUUTYGAGAGAAAATGGAGAGAGT
                                83 84
UTYGAAUUUAGGAGATAGGAGGYGTATTTTUUUUATGUAUUUATGGATUAYGUUUUYGUU
    85 86 87 88
UUUAUATTUUUUUYGTAGGAGGUAAGAUUTGAGYGUTTAUTUAYGTGGUTUUUUUATUYG
                          89 90 91 92
TTUYGYGTUUATUTUUAAGAUTGUUTUAUAGGGAUUUUUAGGAGGUTUYGAAUUATUUA
    93 94 95
GUTTTUTGTUAUYGUYGUYGUUAUUAGYGTTGTGAAUUTUTGAUUUTYGYGGUTUTGYGT
                        96 97 98 99 100 101 102
UUATTUTUAGGTAUTGAAAGTTTTUYGGGUTUTTUYGUAUUYGYGGATGTGGYGAAGUYG
                                                  103 104 105 106 107 108
YGGGGUAGUTUYGUTYGYGUTUUAGTYGUAGGATGTUUTTGAUYGAGAAGGGGGTGGAG
109 110 111 112 113 114
GTGAYGGGGUTUAGUAGUATUUYGAAGGYGGATGGGGYGGGGUYGAGGAGGTUYGGGTG
        115 116 117 118 119 120
AGGAGYGGUAUUUTGAAUTTUUYGTUTTGTYGUTGUAGGUUUYGUAGAUAGAUUUAAGU
          121 122 123 124
TUTGGGAUAGAYGUUUAGYGTUUUAGAUAGYGUUTTUUTUTGGGUUATGUTGGTAGGUU
                      125 126 127
YGGGTUUAGGGUYGGGTGAYGAGAUYGTAGUUUUUUATTGGTTUTYGUAGAAAUUAYGT
128 129 130 131 132 133
GUTAGUTUTGUAUUTTUUTUUUUUAGYGUTTTUTTUUYGYGUUYGTYGAATUUTUTUUAGT
                                                    134 135 136 137 138
UUUUAGTYGUUTTUUTGGGAGGTTTGGTTYGTATTGAYGGTTUTUAAAGUAAAGAAAAYGT
              139 140 141 142
GGAGAGAUAGTGUTGYGYGYGGUTTGGATUTUYGTUAATAYGGTAUTAGYGGGAGUTTU
                          143 144 145 146 147 148
YGAGGTUAAUUTAGGGTUUAYGGGGAUUUTUUTAGTGGAUATGYGGGGATTGGAGGTGGA
149 150 151
GTUUTTGUTGTGUAAUUYGATUYGGTUUATUUAGUUTGGTUTTAGUTUAAUTUTGUATTUT
                                  152 153
TATGGYGGAGGAAAGUTAAGATAGAGAUAGUTGGGAUUYGYGGAATTTGAGGYGYGYGU
          154 155 156 157 158 159
TGGGATUTTAYGTGYGUAGAGGUYGGAUYGUAGAGTAGAAGTGAGTUTTGAUUUAUUAGG
                    160 161 162 163
GUUTATATTGAGATGGAAAUUTUTUTUTTTGAAUAGAATUUAGGTUTGYGGAGTUUYGAU
                                                                                                164 165
UYGUUAUTTUUTAGGAGUAGUTTUTUAGUUUUAGGAUYGAGGAATGGGTGTGGGGGUTAU
  166 167
ATGTGGGGAGUUUTYGAUUTUTTAAAGGGUTUYGYGAATGGTGGGGGAGAUTGGAAGGUA
                            168 169 170
ATUTUTGYGGUUYGYGUAAAGTGGTUAGAGGYGGGAGTGGGGGTGGGGGGGGGTGGGUTT
              171 172 173 174
UYGTYGUUUTTGGYGTUUUUAYGTTUTTTTTUTTUUTUTUUTTTATUUTUUTTAAAGYGGAT
175 176 177 178 179
TGUUTGGAGYGTGUUAGUTTGTGTGYGYGTATAAATGYGGYGUUYGUYGAAGGGUTGGGA
                  180 181 182 183 184 185 186
AGATTTGAUTYGUATTAGATUTUUAGAAAAGGUUUTTGGTTAYGGYGGGUAUYGAGTAAA
                    187 188 189 190
TGGYG
      191
```

The reverse complementary sequence of SEQ ID NO: 1 is shown is SEQ ID NO: 3 as follows:

CGCCATTTACTCGGTGCCCGCCGTAACCAAGGGCCTTTTCTGGAGATCTAATGCGAGTCAAATC
TTCCCAGCCCTTCGGCGGGCGCCGCATTTATACGCGCACACAAGCTGGCACGCTCCAGGCAATC
CGCTTTAAGGAGGATAAAGGAGAGGAAGAAAAAGAACGTGGGGACGCCAAGGGCGACGGAA
GCCCACCCCCCCCCACCCCCACTCCCGCCTCTGACCACTTTGCGCGGGCCGCAGAGATTGCCTT
CCAGTCTCCCCCACCATTCGCGGAGCCCTTTAAGAGGTCGAGGGCTCCCCACATGTAGCCCCCA
CACCCATTCCTCGGTCCTGGGGCTGAGAAGCTGCTCCTAGGAAGTGGCGGGTCGGGACTCCGC
AGACCTGGATTCTGTTCAAAGAGAGAGGTTTCCATCTCAATATAGGCCCTGGTGGGTCAAGACT
CACTTCTACTCTGCGGTCCGGCCTCTGCGCACGTAAGATCCCAGCGCGCGCCTCAAATTCCGCG
GGTCCCAGCTGTCTCTATCTTAGCTTTCCTCCGCCATAAGAATGCAGAGTTGAGCTAAGACCAG
GCTGGATGGACCGGATCGGGTTGCACAGCAAGGACTCCACCTCCAATCCCCGCATGTCCACTA
GGAGGGTCCCCGTGGACCCTAGGTTGACCTCGGAAGCTCCCGCTAGTACCGTATTGACGGAGA
TCCAAGCCGCGCGCAGCACTGTCTCTCCACGTTTTCTTTGCTTTGAGAACCGTCAATACGAACC
AAACCTCCCAGGAAGGCGACTGGGGACTGGAGAGGATTCGACGGGCGCGGGAAGAAAGCGCT
GGGGGAGGAAGGTGCAGAGCTAGCACGTGGTTTCTGCGAGAACCAATGGGGGGCTACGGTCTC
GTCACCCGGCCCTGGACCCGGGCCTACCAGCATGGCCCAGAGGAAGGCGCTGTCTGGGACGCT
GGGCGTCTGTCCCAGAGCTTGGGTCTGTCTGCGGGGCCTGCAGCGACAAGACGGGAAGTTCAG
GGTGCCGCTCCTCACCCGGACCTCCTCGGCCCCGCCCCATCCGCCTTCGGGATGCTGCTGAGCC
CCGTCACCTCCACCCCCTTCTCGGTCAAGGACATCCTGCGACTGGAGCGCGAGCGGAGCTGCCC
CGCGGCTTCGCCACATCCGCGGGTGCGGAAGAGCCCGGAAAACTTTCAGTACCTGAGAATGGA
CGCAGAGCCGCGAGGGTCAGAGGTTCACAACGCTGGTGGCGGCGGCGGTGACAGAAAGCTGG
ATGGTTCGGAGCCTCCTGGGGGTCCCTGTGAGGCAGTCTTGGAGATGGACGCGGAACGGATGG
GGGAGCCACGTGAGTAAGCGCTCAGGTCTTGCCTCCTACGGGGGGAATGTGGGGGCGGGGGCG
TGATCCATGGGTGCATGGGGAAAATACGCCTCCTATCTCCTGGGTTCGAGACTCTCTCCATTTT
CTCTCGAGGAGTCCAAAAAAAGGCCTCTCGAATCCCATTCCCATCCTCTCCTGACACCCAGTGT
GCTTCACACACCCCTAACGAAAGTAAGGCCCTGAATCTCAAGGATTCACTTTTCCCAAATCCGC
TCCTCTGATTCCCACAAACGCATCGCTCACACTCGCAGCCACAGTGCCCAGAACCTCCACCTTC
TCATCTCCACAGCGAAACCCTCCGCAGTGTCTTAGCCGTGCCAGTCACCACCGCCCTTCCCTAC
CATCACCTGGTGTCTCTCCACCCCCAAACCGTTTGCAAAAGCGTGTGGCCATCAAAGCAGTGGA
ACCCTCGTGGCGCTGAGGTCACTCCTAACTGGGCCCTTCTTGCTGGTCTGGTCGGGGGACCCC
CCCATTCCTCCTCATGGATTTTTCTTCTGGAAGACGAAAGTTGGGGACCGGGTGGCGATTGAGT
CTCCCGAAGTGGAAGGCCCCGTTGGCGCAGGATTCTCCGCGTCAGGGAGAACGTTAAAGGTTG
CCAGGGTTTTCTCTGCCCTGGGACACGGTTCTGGCCGAGTCCTGTCGCAGGTATCCAAAGGCGC
AACCGTGCGCGCCGGGCTGCGAGGCCCACGCACCTGTGTCCAGCCCGCTGGAAGGGAAGGGGC
TGGCGGGGAGTTGTGTTGCGTCGATCCGGGAGTTGTGTTGTGTCGATCCGGAGGCTTCAGCTGG
AGAGGCCCGATATTCCAAGGGATGAAAACGCCACCTTTTTCTCTCCAAACCTCAGCACCTCACT
CGGCATTCTCAACGCAGGGGCCCTGAGTAGCGCCCGCCAGGGCAGGCCTCCTCTGCCAGACTG
TGCCATGGCGCCTCCATTCAAGGCGCGAACGAAAGAAATAAACCAAGGGGCTAGATCGGAGTT
ATTTACCGGCCTGCAGGCCGGCTACTCACTAACTCCAGAAGCCTAAGAACATTACCTGCTGTGT
CCGCGAAGAAGCTGGTAGGTTTTCTGCAGCGGATTCTAAACTCCCTCGGCGCTGCTCAGCCAAC

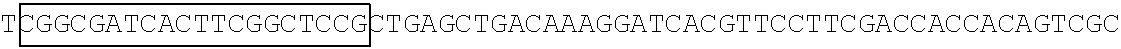

CTCGCAGCCGATGGTATTTGCGGCGCCTTCCTTCTCCCACTGGAAACCAAATCCTGGGAGCGTC
TCTTCTGCGGCAGCAGTGGACGCCGCCGAGGAGGGACCCGAGGCCGGACCCGAGGCCGGCTCC
AGGAACTGAGCCCAAGGCCGCGGTAGCGATCCG

The bisulfite treated sequence of SEQ ID NO: 3 is shown in SEQ ID NO: 4 (Y represents C or U) as follows:

YGUUATTTAUTYGGTGUUYGUYGTAAUUAAGGGUUTTTTUTGGAGATUTAATGYGAGTUAAA
TUTTUUUAGUUUTTYGGYGGGYGUYGUATTTATAYGYGUAUAUAAGUTGGUAYGUTUUAGGU
AATGYGUTTTAAGGAGGATAAAGGAGAGGAAGAAAAAGAAYGTGGGGAYGUUAAGGGYGAY
GGAAGUUUAUUUUUUUUUAUUUUUAUTUUYGUUTUTGAUUAUTTTGYGYGGGUYGUAGAGA
TTGUUTTUUAGTUTUUUUUAUUATTYGYGGAGUUUTTTAAGAGGTYGAGGGUTUUUUAUATG
TAGUUUUAUAUUUATTUUTYGGTUUTGGGGUTGAGAAGUTGUTUUTAGGAAGTGGYGGGTY
GGGAUTUYGUAGAUUTGGATTUTGTTUAAAGAGAGAGGTTTUUATUTUAATATAGGUUUTGG
TGGGTUAAGAUTUAUTTUTAUTUTGYGGTUYGGUUTUTGYGUAYGTAAGATUUUAGYGYGYG
UUTUAAATTUYGYGGGTUUUAGUTUTUTUTATUTTAGUTTUUTUYGUUATAAGAATGUAGAG
TTGAGUTAAGAUUAGGUTGGATGGAUYGGATYGGGTTGUAUAGUAAGGAUTUUAUUTUUAAT
UUUYGUATGTUUAUTAGGAGGGTUUUYGTGGAUUUTAGGTTGAUUTYGGAAGUTUUYGUTAG
TAUYGTATTGAYGGAGATUUAAGUYGYGYGUAGUAUTGTUTUTUUAYGTTTTUTTTGUTTTGA
GAAUYGTUAATAYGAAUUAAAUUTUUUAGGAAGGYGAUTGGGGAUTGGAGAGGATTYGAYG
GGYGYGGGAAGAAAGYGUTGGGGGAGGAAGGTGUAGAGUTAGUAYGTGGTTTUTGYGAGAA
UUAATGGGGGGUTAYGGTUTYGTUAUUYGGUUUTGGAUUYGGGUUTAUUAGUATGGUUUAG
AGGAAGGYGUTGTUTGGGAYGUTGGGYGTUTGTUUUAGAGUTTGGGTUTGTUTGYGGGGUUT
GUAGYGAUAAGAYGGGAAGTTUAGGGTGUYGUTUUTUAUUYGGAUUTUUTYGGUUUYGUUU
UATUYGUUTTYGGGATGUTGUTGAGUUUYGTUAUUTUUAUUUUUTTUTYGGTUAAGGAUATU
UTGYGAUTGGAGYGYGAGYGGAGUTGUUUYGYGGUTTYGUUAUATUYGYGGGTGYGGAAGA
GUUYGGAAAAUTTTUAGTAUUTGAGAATGGAYGUAGAGUYGYGAGGGTGAGAGGTTUAUAA
YGUTGGTGGYGGYGGYGGTGAUAGAAAGUTGGATGGTTYGGAGUUTUUTGGGGGTUUUTGTG
AGGUAGTUTTGGAGATGGAYGYGGAAYGGATGGGGGAGUUAYGTGAGTAAGYGUTUAGGTUT
TGUUTUUTAYGGGGGGAATGTGGGGGYGGGGGYGTGATUUATGGGTGUATGGGGAAAATAYG
UUTUUTATUTTUUTGGGTTYGAGAUTUTUTUUATTTTUTUTYGAGGAGTUUAAAAAAAGGUUTU
TYGAATUUUATTUUUATUUTUTUUTGAUAUUUAGTGTGUTTUAUAUAUUUUTAAYGAAAGTA
AGGUUUTGAATUTUAAGGATTUAUTTTTUUUAAATUYGUTUUTUTGATTUUUAUAAAYGUAT
YGUTUAUAUTYGUAGUUAUAGTGUUUAGAAUUTUUAUUTTUTUATUTUUAUAGYGAAAUUUT
UYGUAGTGTUTTAGUYGTUUAGTUAUUAUYGUUUTTUUUTAUUATUAUUTGGTGTUTUTUU
AUUUUUAAAUYGTTTGUAAAAGYGTGTGGUUATUAAAGUAGTGGAAUUUTYGTGGYGUTGAG
GTUAUTUUTAAUTGGGUUUTTTUTTGUTGGTUTGGTYGGGGGAUUUUUUUATTUUTUUTUATG
GATTTTTUTTUTGGAAGAYGAAAGTTGGGGAUYGGGTGGYGATTGAGTUTUUYGAAGTGGAA
GGUUUYGTTGGYGUAGGATTUTUYGYGTUAGGGAGAAYGTTAAAGGTTGUUAGGGTTTTUTU
TGUUUTGGGAUAYGGTTUTGGUYGAGTUUTGTYGUAGGTATUUAAAGGYGUAAUYGTGYGYG
UYGGGUTGYGAGGUUUAYGUAUUTGTGTUUAGUUYGUTGGAAGGGAAGGGGUTGGYGGGGA
GTTGTGTTGYGTYGATUYGGGAGTTGTGTTGTGTYGATUYGGAGGUTTUAGUTGGAGAGGUUY
GATATTUUAAGGGATGAAAAYGUUAUUTTTTTUTUTUUAAAUGTUAGUAUUTUATYGGUAT
TUTUAAYGUAGGGGUUUTGAGTAGYGUUYGUUAGGGUAGGUUTUUTUTGUUAGAUTGTGUU
ATGGYGUUTUUATTUAAGGYGYGAAYGAAAGAAATAAAUUAAGGGGUTAGATYGGAGTTATT
TAUYGGUUTGUAGGUYGGUTAUTUAUTAAUTUUAGAAGUUTAAGAAUATTAUUTGUTGTGTU
YGYGAAGAAGUTGGTAGGTTTTUTGUAGYGGATTUTAAAUTUUUTYGGYGUTGUTUAGUUAA

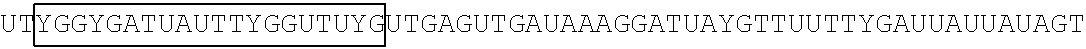

YGUUTYGUAGUYGATGGTATTTGYGGYGUUTTUUTTUTUUUAUTGGAAAUUAAATUUTGGGA
GYGTUTUTTUTGYGGUAGUAGTGGAYGUYGUYGAGGAGGGAUUYGAGGUYGGAUUYGAGGU
YGGUTUUAGGAAUTGAGUUUAAGGUYGYGGTAGYGATUYG

### EXAMPLE 2

### Difference in Methylation of STAMP-EP8 CpG Sites Between Tumor Cells and Non-Tumor Cells—Bisulfite Sequencing PCR (BSP)

Bisulfite Sequencing PCR comprises the following steps:

1. Genomic DNA was extracted from lung cancer cell line A549 and normal lung cell line MRCS;

2. The extracted genomic DNA of lung cancer cell line A549 and normal lung cell line MRCS were treated with bisulfite and used as templates for subsequent PCR amplification;

3. Primers (SEQ ID NO: 5-14; Table 1)were designed according to SEQ ID NO: 1 and used for amplification;

4. After PCR amplification, 2% agarose gel electrophoresis was used to detect the specificity of the PCR fragments. The target fragments were recovered by gel cutting and connected to T vector which was transformed into competent Escherichia coli. The bacteria were spread on plate, and clones were selected the next day and sequenced. More than ten clones were selected for each fragment for Sanger sequencing.

TABLE 1

| Primer Name | 5′-3′ Primer Sequence | Detected CpG site No. |
|---|---|---|
| STAMP-EP8-Sanger- | ATGAAGTTTATYGAAGGTGTTT | CpG 001-040 of SEQ ID |
| Primer-F | (SEQ ID NO: 5) | NO: 1 |
| STAMP-EP8-Sanger- | TTTCTCTCCAAACCTCAACACCTC | |
| Primer-R | (SEQ ID NO: 6) | |
| STAMP-EP8-Sanger- | GAGGTGTTGAGGTTTGGAGAGAAA | CpG 041-077 of SEQ ID |
| Primer-F | (SEQ ID NO: 7) | NO: 1 |
| STAMP-EP8-Sanger- | AACCTCCACCTTCTCATCTCCACA | |
| Primer-R | (SEQ ID NO: 8) | |
| STAMP-EP8-Sanger- | TGTGGAGATGAGAAGGTGGAGGTT | CpG 078-114 of SEQ ID |
| Primer-F | (SEQ ID NO: 9) | NO: 1 |
| STAMP-EP8-Sanger- | ATACTACTAAACCCCRTCACCT | |
| Primer-R | (SEQ ID NO: 10) | |
| STAMP-EP8-Sanger- | ATYGAGAAGGGGGTGGAGGTGA | CpG 115-153 of SEQ ID |
| Primer-F | (SEQ ID NO: 11) | NO: 1 |
| STAMP-EP8-Sanger- | AACTAAAACCAAACTAAATAAAC | |
| Primer-R | (SEQ ID NO: 12) | |
| STAMP-EP8-Sanger- | GTTTATTTAGTTTGGTTTTAGTT | CpG 154-191 of SEQ ID |
| Primer-F | (SEQ ID NO: 13) | NO: 1 |
| STAMP-EP8-Sanger- | TAATAACCTTTATACCACACTAC | |
| Primer-R | (SEQ ID NO: 14) | |

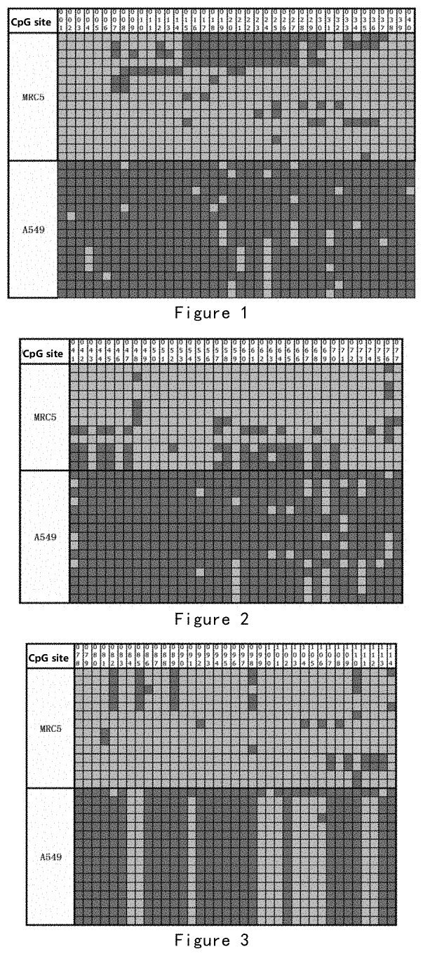

FIG. 1 shows BSP assay for methylation levels of the methylation sites 001 to 040 in SEQ ID NO:1 region in lung cancer cells and normal lung cells, which indicates that the methylation level of STAMP-EP8 of lung cancer cells is significantly higher than that of normal lung cells.

FIG. 2 shows BSP assay for methylation levels of the methylation sites 041 to 077 in SEQ ID NO:1 region in lung cancer cells and normal lung cells, which indicates that the methylation level of STAMP-EP8 of lung cancer cells is significantly higher than that of normal lung cells.

FIG. 3 shows BSP assay for methylation levels of the methylation sites 078 to 114 in SEQ ID NO:1 region in lung cancer cells and normal lung cells, which indicates that the methylation level of STAMP-EP8 of lung cancer cells is significantly higher than that of normal lung cells.

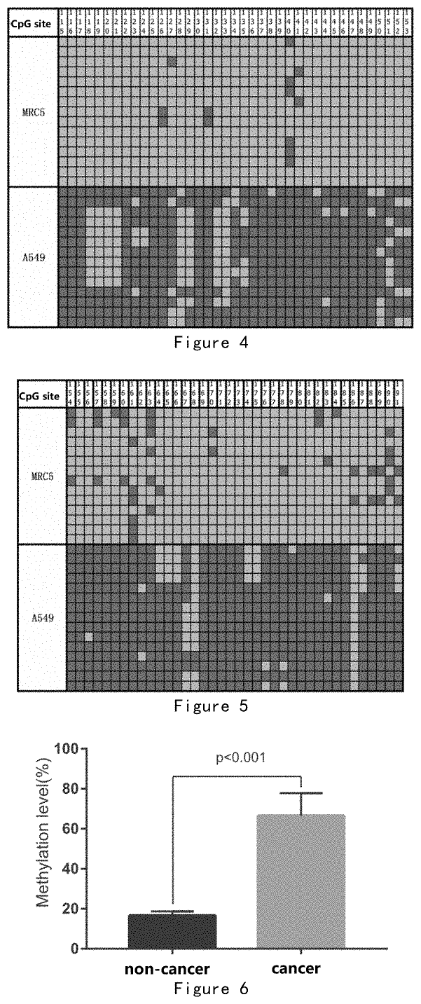

FIG. 4 shows BSP assay for methylation levels of the methylation sites 115 to 153 in SEQ ID NO:1 region in lung cancer cells and normal lung cells, which indicates that the methylation level of STAMP-EP8 of lung cancer cells is significantly higher than that of normal lung cells.

FIG. 5 shows BSP assay for methylation levels of the methylation sites 154 to 191 in SEQ ID NO:1 region in lung cancer cells and normal lung cells, which indicates that the methylation level of STAMP-EP8 of lung cancer cells is significantly higher than that of normal lung cells.

### EXAMPLE 3

### Difference in Methylation of STAMP-EP8 CpG Sites Between Tumor Cells and Non-Tumor Cells—Pyrosequencing

Pyrosequencing comprises the following steps:

1. Clinical samples: paracancerous/non-cancer were used as the control group, and cancer tissue samples were used as the experimental group;

2. DNA extraction: DNA was extracted from the experimental group and the control group respectively. Phenol-chloroform extraction method was used in this experiment, which is not limited thereto;

3. Bisulfite treatment: the extracted DNA samples were treated with bisulfite and the procedures were strictly followed. EZ DNA Methylation-Gold Kit (ZYMO Research, Cat # D5006) was used in this experiment, which is not limited thereto;

4. Primer design: PCR primers and pyrosequencing primers were designed according to the characteristics of SEQ ID NO: 1 of STAMP-EP8 sequence. The methylation values of STAMP-EP8 were detected as the methylation values of CpG sites 021-024. The PCR primers sequences, pyrosequencing primers sequences, and the pyrosequencing detecting sequences and the detected sites are shown as SEQ ID NO: 15-18 (Table 2); PCR amplification and agarose gel electrophoresis: The bisulfite treated samples were used as templates for PCR amplification. The specificity of PCR amplification was identified by agarose gel electrophoresis of the amplified products;

5. PCR amplification and agarose gel electrophoresis: The bisulfite treated samples were used as templates for PCR amplification. The specificity of PCR amplification was identified by agarose gel electrophoresis of the amplified products;

6. Pyrosequencing: Pyro Mark Q96 ID pyrosequencing instrument (QIAGEN) was used for sequencing, and the procedures in instructions were strictly followed;

7. Calculation of STAMP-EP8 methylation value: pyrosequencing can detect the methylation value of each site in the target region, respectively, and the average methylation value of all sites were calculated as the STAMP-EP8 methylation value in the sample;

8. Results analysis: the methylation value of STAMP-EP8 was compared between the paracancerous/non-cancer control group and the cancer experimental group.

TABLE 2

| Primer Name | 5′-3′ sequence | Detected CpG site No. |
|---|---|---|
| STAMP-EP8-Pyroseq- | GAGGYGATTGTGGTGGTYGAAGGAA | The primer pair detects |
| Primer-F | (SEQ ID NO: 15) | CpG 021-024 of SEQ |
| STAMP-EP8-Pyroseq- | AAACTCCCTCRACRCTACTCAACCAACTC | ID NO: 1 |
| Primer-R (with 5′-Biotin | (SEQ ID NO: 16) | |
| modification) | | |
| STAMP-EP8-Pyroseq- | GAAGGAAYGTGATTTTTTGTTAGTTTAG | |
| Primer-Seq | (SEQ ID NO: 17) | |
| pyrosequencing detecting | YGGAGTYGAAGTGATYGTYGAGTTGGTTG | |
| sequences | AGTAGYGTYGA (SEQ ID NO: 18) | |

### EXAMPLE 4

### Leukemia Clinical Sample Assay—Pyrosequencing

STAMP-EP8 methylation value between the control group of 8 non-leukemia bone marrow smear clinical samples and the experimental group of 8 leukemia bone marrow smear clinical samples was compared according to the pyrosequencing in Example 3.

The result in FIG. 6 shows that, in clinical samples of leukemia, the methylation value of STAMP-EP8 in the experimental group was significantly higher than that of non-cancer tissues.

### EXAMPLE 5

### Breast Cancer Clinical Sample Assay—Pyrosequencing

STAMP-EP8 methylation value between the control group of 5 cases of breast cancer paracancerous clinical samples and the experimental group of 5 cases of breast cancer clinical samples was compared according to the pyrosequencing in Example 3.

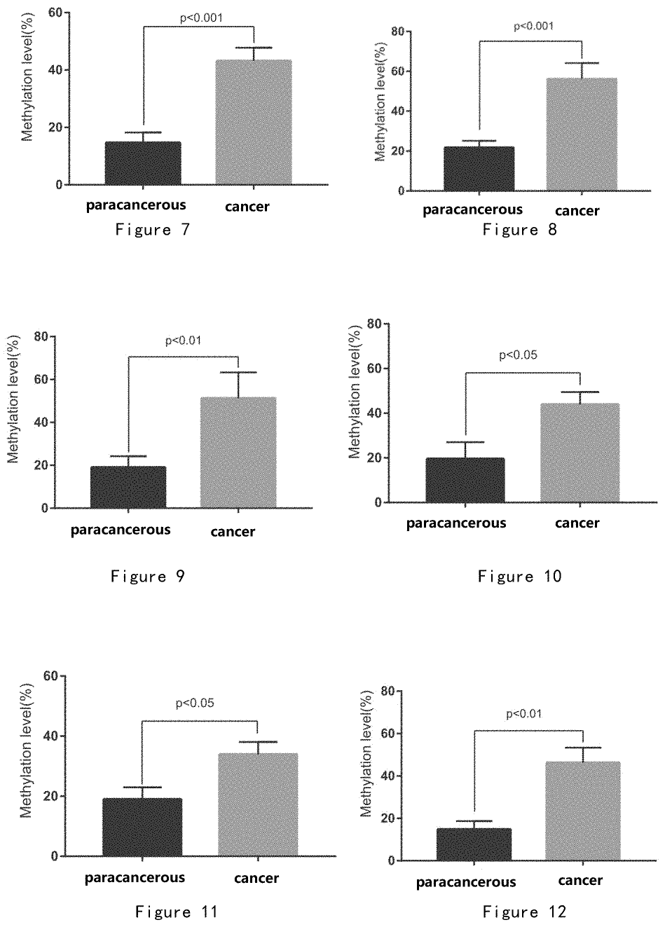

The result in FIG. 7 shows that, in clinical samples of breast cancer, the methylation value of STAMP-EP8 in the experimental group was significantly higher than that of paracancerous tissues.

### EXAMPLE 6

### Colorectal Cancer Clinical Sample Assay—Pyrosequencing

STAMP-EP8 methylation value was analyzed on the control group of 8 cases of colorectal cancer paracancerous clinical samples and the experimental group of 8 cases of colorectal cancer clinical samples according to the pyrosequencing in Example 3.

The result in FIG. 8 shows that, in clinical samples of colorectal cancer, the methylation value of STAMP-EP8 in the experimental group was significantly higher than that of paracancerous tissues.

### EXAMPLE 7

### Esophageal Cancer Clinical Sample Assay—Pyrosequencing

STAMP-EP8 methylation value was analyzed on the control group of 10 cases of esophageal cancer paracancerous clinical samples and the experimental group of 10 cases of esophageal cancer clinical samples according to the pyrosequencing in Example 3.

The result in FIG. 9 shows that, in clinical samples of esophageal cancer, the methylation value of STAMP-EP8 in the experimental group was significantly higher than that of paracancerous tissues.

### EXAMPLE 8

### Liver Cancer Clinical Sample Assay—Pyrosequencing

STAMP-EP8 methylation value was analyzed on the control group of 8 cases of liver cancer paracancerous clinical samples and the experimental group of 8 cases of liver cancer clinical samples according to the pyrosequencing in Example 3.

The result in FIG. 10 shows that, in clinical samples of liver cancer, the methylation value of STAMP-EP8 in the experimental group was significantly higher than that of paracancerous tissues.

### EXAMPLE 9

### Lung Cancer Clinical Sample Assay—Pyrosequencing

STAMP-EP8 methylation value was analyzed on the control group of 4 cases of lung cancer paracancerous clinical samples and the experimental group of 4 cases of lung cancer clinical samples according to the pyrosequencing in Example 3.

The result in FIG. 11 shows that, in clinical samples of lung cancer, the methylation value of STAMP-EP8 in the experimental group was significantly higher than that of paracancerous tissues.

### EXAMPLE 10

### Pancreatic Cancer Clinical Sample Assay—Pyrosequencing

STAMP-EP8 methylation value was analyzed on the control group of 4 cases of pancreatic cancer paracancerous clinical samples and the experimental group of 4 cases of pancreatic cancer clinical samples according to the pyrosequencing in Example 3.

The result in FIG. 12 shows that, in clinical samples of pancreatic cancer, the methylation value of STAMP-EP8 in the experimental group was significantly higher than that of paracancerous tissues.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1.-6. (canceled)

7. A method for detecting tumor, wherein said method comprising:
detecting the modification on the CpG site(s) of a polynucleotide by a tumor detection agent or kit, if the hypermethylation of a subject is detected, the subject can be identified as having a high-risk of tumor; said polynucleotide comprises: (a) a polynucleotide with a nucleotide sequence as shown in SEQ ID NO: 1; (b) a fragment of the polynucleotide of (a), having at least one CpG site with modification; and/or (c) a nucleic acid complementary to the polynucleotide or fragment of (a) or (b).

8. The method according to claim 7, wherein, the tumors comprise: hematologic cancers such as leukemia, lymphoma, multiple myeloma; gynecological and reproductive system tumors such as breast cancer, ovarian cancer, cervical cancer, vulvar cancer, testicular cancer, prostate cancer, penile cancer; digestive system tumors such as esophageal cancer, gastric cancer, colorectal cancer, liver cancer, pancreatic cancer, bile duct and gallbladder cancer;
respiratory system tumors such as lung cancer, pleuroma; nervous system tumors such as glioma, neuroblastoma, meningioma; head and neck tumors such as oral cancer, tongue cancer, laryngeal cancer, nasopharyngeal cancer; urinary system tumors such as kidney cancer, bladder cancer, skin and other systems tumors such as skin cancer, melanoma, osteosarcoma, liposarcoma, thyroid cancer.

9. The method according to claim 7, wherein samples of the tumor comprise: tissue samples, paraffin embedded samples, blood samples, pleural effusion samples, and alveolar lavage fluid samples, ascites and lavage fluid samples, bile samples, stool samples, urine samples, saliva samples, sputum samples, cerebrospinal fluid samples, cell smear samples, cervical scraping or brushing samples, tissue and cell biopsy samples.

10. A method of preparing a tumor detection agent, comprising: providing a and designing a detection agent for specifically detecting a target sequence which is the full length or fragment of the polynucleotide; wherein said polynucleotide comprises: (a) a polynucleotide with a nucleotide sequence as shown in SEQ ID NO: 1; (b) a fragment of the polynucleotide of (a), having at least one CpG site with modification; and/or (c) a nucleic acid complementary to the polynucleotide or fragment of (a) or (b).

11. (canceled)

12. The method according to claim 10, wherein the detection agent specifically detects a gene sequence containing the target sequence, and the gene sequence comprises gene panels or gene groups.

13. The method according to claim 10, wherein the detection agent comprises a primer or a probe.

14. (canceled)

15. A detection kit, comprising:
container(s) and a detection agent in the container(s)); said detection agent for specifically detecting the modification of the CpG(s) of the target sequence which is the full length or fragment of the polynucleotide; wherein said polynucleotide comprises: (a) a polynucleotide with a nucleotide sequence as shown in SEQ ID NO: 1; (b) a fragment of the polynucleotide of (a), having at least one CpG site with modification; and/or (c) a nucleic acid complementary to the polynucleotide or fragment of (a) or (b).

16. A method of detecting the methylation profile of a sample in vitro, comprising:
(i) providing the sample and extracting nucleic acid; and
(ii) detecting modification on CPG site(s) of a target sequence in the nucleic acid of (i), wherein the target sequence is the polynucleotide comprises (a) a polynucleotide with a nucleotide sequence as shown in SEQ ID NO: 1; (b) a fragment of the polynucleotide of (a), having at least one CpG site with modification; and/or (c) a nucleic acid complementary to the polynucleotide or fragment of (a) or (b).

17. The method according to claim 16, wherein, in step (ii), the analysis methods comprise pyrosequencing, bisulfite conversion sequencing, method using methylation chip, qPCR, digital PCR, second generation sequencing, third generation sequencing, whole genome methylation sequencing, DNA enrichment detection, simplified bisulfite sequencing technology, HPLC, MassArray, methylation specific PCR, or their combination, as well as in vitro detection and in vivo tracer detection for the combined gene group of partial or all of the methylation sites in the sequence shown in SEQ ID NO: 1.

18. The method according to claim 16, wherein, step (ii) comprises:
(1) treating the product of (i) to convert unmodified cytosine into uracil; and
(2) analyzing the modification of the target sequence in the nucleic acid treated by (1).

19. The method according to claim 7, wherein said tumor detection agent or kit is specifically detect the polynucleotide, or the panel or gene group containing the polynucleotide.

20. The method according to claim 7, wherein for (b), the fragment of the polynucleotide contains: bases at positions 204 to 223 of SEQ ID NO: 1 or 2; bases at positions 1 to 478 of SEQ ID NO: 1 or 2; bases at positions 513 to 1040 of SEQ ID NO: 1 or 2; bases at positions 1082 to 1602 of SEQ ID NO: 1 or 2; bases at positions 1621 to 2117 of SEQ ID NO: 1 or 2; or bases at positions 2160 to 2700 of SEQ ID NO: 1 or 2.

21. The method according to claim 20, wherein said tumor detection agent or kit comprises primers of probes.

22. The method according to claim 21, wherein said primers are:
primers shown in SEQ ID NO: 15 and 16;
primers shown in SEQ ID NO: 5 and 6;
primers shown in SEQ ID NO: 7 and 8;
primers shown in SEQ ID NO: 9 and 10;
primers shown in SEQ ID NO: 11 and 12; or
primers shown in SEQ ID NO: 13 and 14.

23. The method according to claim 7, wherein said modification comprises 5-methylation, 5-hydroxymethylation, 5-formylcytosine or 5-carboxylcytosine.

24. The method according to claim 13, wherein the primer is:
the primers shown in SEQ ID NO: 5 and 6;
the primers shown in SEQ ID NO: 7 and 8;
the primers shown in SEQ ID NO: 9 and 10;
the primers shown in SEQ ID NO: 11 and 12;
the primers shown in SEQ ID NO: 13 and 14; or
the primers shown in SEQ ID NO: 15 and 16.

25. The detection kit according to claim 15, wherein the primers are:
the primers shown in SEQ ID NO: 15 and 16;
the primers shown in SEQ ID NO: 5 and 6;
the primers shown in SEQ ID NO: 7 and 8;
the primers shown in SEQ ID NO: 9 and 10;
the primers shown in SEQ ID NO: 11 and 12; or
the primers shown in SEQ ID NO: 13 and 14.

26. The method according to claim 17, wherein the modification includes 5-methylation, 5-hydroxymethylation, 5-formylcytosine or 5-carboxylcytosine.

27. The method according to claim 17, wherein treating the nucleic acid of step (i) with bisulfite.

28. An isolated polynucleotide, wherein, the polynucleotide is converted from an original polynucleotide, said original polynucleotide comprises: (a) a polynucleotide with a nucleotide sequence as shown in SEQ ID NO: 1; (b) a fragment of the polynucleotide of (a), having at least one CpG site with modification; and/or (c) a nucleic acid complementary to the polynucleotide or fragment of (a) or (b); and as compared with the sequence of the original polynucleotide, cytosine C of the CpG site(s) with modification is unchanged, and the unmodified cytosine is converted into T or U in the converted polynucleotide.

29. The isolated polynucleotide according to claim 28, wherein treating the nucleic acid of the original polynucleotide with bisulfite to obtain the converted polynucleotide.

30. The isolated polynucleotide according to claim 28, wherein the isolated polynucleotide comprises: (d) a polynucleotide with a nucleotide sequence as shown in SEQ ID NO: 2 or 4; or (e) a fragment of the polynucleotide of (d), having at least one CpG site with modification.

31. The polynucleotide according to claim 30, wherein, for (e), the fragment of the polynucleotide contains:
bases at positions 204 to 223 of SEQ ID NO: 1 or 2;
bases at positions 1 to 478 of SEQ ID NO: 1 or 2;
bases at positions 513 to 1040 of SEQ ID NO: 1 or 2;
bases at positions 1082 to 1602 of SEQ ID NO: 1 or 2;
bases at positions 1621 to 2117 of SEQ ID NO: 1 or 2;
bases at positions 2160 to 2700 of SEQ ID NO: 1 or 2;
bases at positions 2478 to 2497 of SEQ ID NO: 4 or 3;
bases at positions 2223 to 2700 of SEQ ID NO: 4 or 3;
bases at positions 1661 to 2188 of SEQ ID NO: 4 or 3;
bases at positions 1099 to 1619 of SEQ ID NO: 4 or 3;
bases at positions 584 to 1080 of SEQ ID NO: 4 or 3; or
bases at positions 1 to 541 of SEQ ID NO: 4 or 3.


SEQUENCE LISTING (18 sequences)

SEQ ID NO: 1 - DNA, 2700 bp, Homo sapiens
cggatcgctaccgcggccttgggctcagttcctggagccggcctcgggtccggcctcgggtccctcctcggcggcgtccactgctgccgcagaagagacgctcccaggatttggtttccagtgggagaaggaaggcgccgcaaataccatcggctgcgaggcgactgtggtggtcgaaggaacgtgatcctttgtcagctcagcggagccgaagtgatcgccgagttggctgagcagcgccgagggagtttagaatccgctgcagaaaacctaccagcttcttcgcggacacagcaggtaatgttcttaggcttctggagttagtgagtagccggcctgcaggccggtaaataactccgatctagccccttggtttatttctttcgttcgcgccttgaatggaggcgccatggcacagtctggcagaggaggcctgccctggcgggcgctactcagggcccctgcgttgagaatgccgagtgaggtgctgaggtttggagagaaaaaggtggcgttttcatcccttggaatatcgggcctctccagctgaagcctccggatcgacacaacacaactcccggatcgacgcaacacaactccccgccagccccttcccttccagcgggctggacacaggtgcgtgggcctcgcagcccggcgcgcacggttgcgcctttggatacctgcgacaggactcggccagaaccgtgtcccagggcagagaaaaccctggcaacctttaacgttctccctgacgcggagaatcctgcgccaacggggccttccacttcgggagactcaatcgccacccggtccccaactttcgtcttccagaagaaaaatccatgaggaggaatgggggggtcccccgaccagaccagcaagaaagggcccagttaggagtgacctcagcgccacgagggttccactgctttgatggccacacgcttttgcaaacggtttgggggtggagagacaccaggtgatggtagggaagggcggtggtgactggcacggctaagacactgcggagggtttcgctgtggagatgagaaggtggaggttctgggcactgtggctgcgagtgtgagcgatgcgtttgtgggaatcagaggagcggatttgggaaaagtgaatccttgagattcagggccttactttcgttaggggtgtgtgaagcacactgggtgtcaggagaggatgggaatgggattcgagaggcctttttttggactcctcgagagaaaatggagagagtctcgaacccaggagataggaggcgtattttccccatgcacccatggatcacgcccccgcccccacattccccccgtaggaggcaagacctgagcgcttactcacgtggctcccccatccgttccgcgtccatctccaagactgcctcacagggacccccaggaggctccgaaccatccagctttctgtcaccgccgccgccaccagcgttgtgaacctctgaccctcgcggctctgcgtccattctcaggtactgaaagttttccgggctcttccgcacccgcggatgtggcgaagccgcggggcagctccgctcgcgctccagtcgcaggatgtccttgaccgagaagggggtggaggtgacggggctcagcagcatcccgaaggcggatggggcggggccgaggaggtccgggtgaggagcggcaccctgaacttcccgtcttgtcgctgcaggccccgcagacagacccaagctctgggacagacgcccagcgtcccagacagcgccttcctctgggccatgctggtaggcccgggtccagggccgggtgacgagaccgtagccccccattggttctcgcagaaaccacgtgctagctctgcaccttcctcccccagcgctttcttcccgcgcccgtcgaatcctctccagtccccagtcgccttcctgggaggtttggttcgtattgacggttctcaaagcaaagaaaacgtggagagacagtgctgcgcgcggcttggatctccgtcaatacggtactagcgggagcttccgaggtcaacctagggtccacggggaccctcctagtggacatgcggggattggaggtggagtccttgctgtgcaacccgatccggtccatccagcctggtcttagctcaactctgcattcttatggcggaggaaagctaagatagagacagctgggacccgcggaatttgaggcgcgcgctgggatcttacgtgcgcagaggccggaccgcagagtagaagtgagtcttgacccaccagggcctatattgagatggaaacctctctctttgaacagaatccaggtctgcggagtcccgacccgccacttcctaggagcagcttctcagccccaggaccgaggaatgggtgtgggggctacatgtggggagccctcgacctcttaaagggctccgcgaatggtgggggagactggaaggcaatctctgcggcccgcgcaaagtggtcagaggcgggagtgggggtgggggggggtgggcttccgtcgcccttggcgtccccacgttctttttcttcctctcctttatcctccttaaagcggattgcctggagcgtgccagcttgtgtgcgcgtataaatgcggcgcccgccgaagggctgggaagatttgactcgcattagatctccagaaaaggcccttggttacggcgggcaccgagtaaatggcg

SEQ ID NO: 2 - DNA, 2700 bp, Artificial Sequence
  polynucleotide treated with bisulfite
  misc_feature (1)..(2700): y is c or u
yggatygutauygygguuttgggutuagttuutggaguygguutygggtuygguutygggtuuutuutyggyggygtuuautgutguyguagaagagaygutuuuaggatttggtttuuagtgggagaaggaaggyguyguaaatauuatyggutgygaggygautgtggtggtygaaggaaygtgatuutttgtuagutuagyggaguygaagtgatyguygagttggutgaguagyguygagggagtttagaatuygutguagaaaauutauuaguttuttygyggauauaguaggtaatgttuttagguttutggagttagtgagtaguygguutguagguyggtaaataautuygatutaguuuuttggtttatttutttygttygyguuttgaatggaggyguuatgguauagtutgguagaggagguutguuutggygggygutautuaggguuuutgygttgagaatguygagtgaggtgutgaggtttggagagaaaaaggtggygttttuatuuuttggaatatyggguututuuagutgaaguutuyggatygauauaauauaautuuyggatygayguaauauaautuuuyguuaguuuuttuuuttuuagygggutggauauaggtgygtggguutyguaguuyggygyguayggttgyguutttggatauutgygauaggautygguuagaauygtgtuuuaggguagagaaaauuutgguaauutttaaygttutuuutgaygyggagaatuutgyguuaaygggguuttuuauttygggagautuaatyguuauuyggtuuuuaautttygtuttuuagaagaaaaatuuatgaggaggaatgggggggtuuuuygauuagauuaguaagaaaggguuuagttaggagtgauutuagyguuaygagggttuuautgutttgatgguuauayguttttguaaayggtttgggggtggagagauauuaggtgatggtagggaagggyggtggtgautgguayggutaagauautgyggagggtttygutgtggagatgagaaggtggaggttutggguautgtggutgygagtgtgagygatgygtttgtgggaatuagaggagyggatttgggaaaagtgaatuuttgagattuaggguuttautttygttaggggtgtgtgaaguauautgggtgtuaggagaggatgggaatgggattygagagguutttttttggautuutygagagaaaatggagagagtutygaauuuaggagataggaggygtattttuuuuatguauuuatggatuayguuuuyguuuuuauattuuuuuygtaggagguaagauutgagyguttautuaygtggutuuuuuatuygttuygygtuuatutuuaagautguutuauagggauuuuuaggaggutuygaauuatuuagutttutgtuauyguyguyguuauuagygttgtgaauututgauuutygyggututgygtuuattutuaggtautgaaagttttuygggututtuyguauuygyggatgtggygaaguygygggguagutuygutygygutuuagtyguaggatgtuuttgauygagaagggggtggaggtgayggggutuaguaguatuuygaaggyggatggggygggguygaggaggtuygggtgaggagygguauuutgaauttuuygtuttgtygutguagguuuyguagauagauuuaagututgggauagayguuuagygtuuuagauagyguuttuututggguuatgutggtagguuygggtuuaggguygggtgaygagauygtaguuuuuuattggttutyguagaaauuaygtgutagututguauuttuutuuuuuagygutttuttuuygyguuygtygaatuututuuagtuuuuagtyguuttuutgggaggtttggttygtattgayggttutuaaaguaaagaaaaygtggagagauagtgutgygygygguttggatutuygtuaatayggtautagygggaguttuygaggtuaauutagggtuuayggggauuutuutagtggauatgyggggattggaggtggagtuuttgutgtguaauuygatuyggtuuatuuaguutggtuttagutuaaututguattuttatggyggaggaaagutaagatagagauagutgggauuygyggaatttgaggygygygutgggatuttaygtgyguagagguyggauyguagagtagaagtgagtuttgauuuauuaggguutatattgagatggaaauututututttgaauagaatuuaggtutgyggagtuuygauuyguuauttuutaggaguaguttutuaguuuuaggauygaggaatgggtgtgggggutauatgtggggaguuutygauututtaaagggutuygygaatggtgggggagautggaagguaatututgygguuygyguaaagtggtuagaggygggagtgggggtgggggggggtggguttuygtyguuuttggygtuuuuaygttutttttuttuututuutttatuutuuttaaagyggattguutggagygtguuaguttgtgtgygygtataaatgyggyguuyguygaagggutgggaagatttgautyguattagatutuuagaaaagguuuttggttayggyggguauygagtaaatggyg

SEQ ID NO: 3 - DNA, 2700 bp, Homo sapiens
cgccatttactcggtgcccgccgtaaccaagggccttttctggagatctaatgcgagtcaaatcttcccagcccttcggcgggcgccgcatttatacgcgcacacaagctggcacgctccaggcaatccgctttaaggaggataaaggagaggaagaaaaagaacgtggggacgccaagggcgacggaagcccacccccccccacccccactcccgcctctgaccactttgcgcgggccgcagagattgccttccagtctcccccaccattcgcggagccctttaagaggtcgagggctccccacatgtagcccccacacccattcctcggtcctggggctgagaagctgctcctaggaagtggcgggtcgggactccgcagacctggattctgttcaaagagagaggtttccatctcaatataggccctggtgggtcaagactcacttctactctgcggtccggcctctgcgcacgtaagatcccagcgcgcgcctcaaattccgcgggtcccagctgtctctatcttagctttcctccgccataagaatgcagagttgagctaagaccaggctggatggaccggatcgggttgcacagcaaggactccacctccaatccccgcatgtccactaggagggtccccgtggaccctaggttgacctcggaagctcccgctagtaccgtattgacggagatccaagccgcgcgcagcactgtctctccacgttttctttgctttgagaaccgtcaatacgaaccaaacctcccaggaaggcgactggggactggagaggattcgacgggcgcgggaagaaagcgctgggggaggaaggtgcagagctagcacgtggtttctgcgagaaccaatggggggctacggtctcgtcacccggccctggacccgggcctaccagcatggcccagaggaaggcgctgtctgggacgctgggcgtctgtcccagagcttgggtctgtctgcggggcctgcagcgacaagacgggaagttcagggtgccgctcctcacccggacctcctcggccccgccccatccgccttcgggatgctgctgagccccgtcacctccacccccttctcggtcaaggacatcctgcgactggagcgcgagcggagctgccccgcggcttcgccacatccgcgggtgcggaagagcccggaaaactttcagtacctgagaatggacgcagagccgcgagggtcagaggttcacaacgctggtggcggcggcggtgacagaaagctggatggttcggagcctcctgggggtccctgtgaggcagtcttggagatggacgcggaacggatgggggagccacgtgagtaagcgctcaggtcttgcctcctacggggggaatgtgggggcgggggcgtgatccatgggtgcatggggaaaatacgcctcctatctcctgggttcgagactctctccattttctctcgaggagtccaaaaaaaggcctctcgaatcccattcccatcctctcctgacacccagtgtgcttcacacacccctaacgaaagtaaggccctgaatctcaaggattcacttttcccaaatccgctcctctgattcccacaaacgcatcgctcacactcgcagccacagtgcccagaacctccaccttctcatctccacagcgaaaccctccgcagtgtcttagccgtgccagtcaccaccgcccttccctaccatcacctggtgtctctccacccccaaaccgtttgcaaaagcgtgtggccatcaaagcagtggaaccctcgtggcgctgaggtcactcctaactgggccctttcttgctggtctggtcgggggacccccccattcctcctcatggatttttcttctggaagacgaaagttggggaccgggtggcgattgagtctcccgaagtggaaggccccgttggcgcaggattctccgcgtcagggagaacgttaaaggttgccagggttttctctgccctgggacacggttctggccgagtcctgtcgcaggtatccaaaggcgcaaccgtgcgcgccgggctgcgaggcccacgcacctgtgtccagcccgctggaagggaaggggctggcggggagttgtgttgcgtcgatccgggagttgtgttgtgtcgatccggaggcttcagctggagaggcccgatattccaagggatgaaaacgccacctttttctctccaaacctcagcacctcactcggcattctcaacgcaggggccctgagtagcgcccgccagggcaggcctcctctgccagactgtgccatggcgcctccattcaaggcgcgaacgaaagaaataaaccaaggggctagatcggagttatttaccggcctgcaggccggctactcactaactccagaagcctaagaacattacctgctgtgtccgcgaagaagctggtaggttttctgcagcggattctaaactccctcggcgctgctcagccaactcggcgatcacttcggctccgctgagctgacaaaggatcacgttccttcgaccaccacagtcgcctcgcagccgatggtatttgcggcgccttccttctcccactggaaaccaaatcctgggagcgtctcttctgcggcagcagtggacgccgccgaggagggacccgaggccggacccgaggccggctccaggaactgagcccaaggccgcggtagcgatccg

SEQ ID NO: 4 - DNA, 2700 bp, Artificial sequence
  polynucleotide treated with bisulfite
  misc_feature (1)..(2700): y is c or u
yguuatttautyggtguuyguygtaauuaaggguuttttutggagatutaatgygagtuaaatuttuuuaguuuttyggygggyguyguatttataygyguauauaagutgguaygutuuagguaatuygutttaaggaggataaaggagaggaagaaaaagaaygtggggayguuaagggygayggaaguuuauuuuuuuuuauuuuuautuuyguututgauuautttgygyggguyguagagattguuttuuagtutuuuuuauuattygyggaguuutttaagaggtygagggutuuuuauatgtaguuuuuauauuuattuutyggtuutggggutgagaagutgutuutaggaagtggygggtygggautuyguagauutggattutgttuaaagagagaggtttuuatutuaatatagguuutggtgggtuaagautuauttutaututgyggtuygguututgyguaygtaagatuuuagygygyguutuaaattuygygggtuuuagutgtututatuttagutttuutuyguuataagaatguagagttgagutaagauuaggutggatggauyggatygggttguauaguaaggautuuauutuuaatuuuyguatgtuuautaggagggtuuuygtggauuutaggttgauutyggaagutuuygutagtauygtattgayggagatuuaaguygygyguaguautgtututuuaygttttutttgutttgagaauygtuaataygaauuaaauutuuuaggaaggygautggggautggagaggattygaygggygygggaagaaagygutgggggaggaaggtguagagutaguaygtggtttutgygagaauuaatggggggutayggtutygtuauuygguuutggauuyggguutauuaguatgguuuagaggaaggygutgtutgggaygutgggygtutgtuuuagaguttgggtutgtutgygggguutguagygauaagaygggaagttuagggtguygutuutuauuyggauutuutygguuuyguuuuatuyguuttygggatgutgutgaguuuygtuauutuuauuuuuttutyggtuaaggauatuutgygautggagygygagyggagutguuuygygguttyguuauatuygygggtgyggaagaguuyggaaaautttuagtauutgagaatggayguagaguygygagggtuagaggttuauaaygutggtggyggyggyggtgauagaaagutggatggttyggaguutuutgggggtuuutgtgagguagtuttggagatggaygyggaayggatgggggaguuaygtgagtaagygutuaggtuttguutuutayggggggaatgtgggggygggggygtgatuuatgggtguatggggaaaatayguutuutatutuutgggttygagautututuuattttututygaggagtuuaaaaaaagguututygaatuuuattuuuatuututuutgauauuuagtgtguttuauauauuuutaaygaaagtaagguuutgaatutuaaggattuauttttuuuaaatuygutuututgattuuuauaaayguatygutuauautyguaguuauagtguuuagaauutuuauuttutuatutuuauagygaaauuutuyguagtgtuttaguygtguuagtuauuauyguuuttuuutauuatuauutggtgtututuuauuuuuaaauygtttguaaaagygtgtgguuatuaaaguagtggaauuutygtggygutgaggtuautuutaautggguuutttuttgutggtutggtygggggauuuuuuuattuutuutuatggatttttuttutggaagaygaaagttggggauygggtggygattgagtutuuygaagtggaagguuuygttggyguaggattutuygygtuagggagaaygttaaaggttguuagggttttututguuutgggauayggttutgguygagtuutgtyguaggtatuuaaaggyguaauygtgygyguygggutgygagguuuayguauutgtgtuuaguuygutggaagggaaggggutggyggggagttgtgttgygtygatuygggagttgtgttgtgtygatuyggagguttuagutggagagguuygatattuuaagggatgaaaayguuauutttttututuuaaauutuaguauutuautygguattutuaayguagggguuutgagtagyguuyguuaggguagguutuututguuagautgtguuatggyguutuuattuaaggygygaaygaaagaaataaauuaaggggutagatyggagttatttauygguutguagguyggutautuautaautuuagaaguutaagaauattauutgutgtgtuygygaagaagutggtaggttttutguagyggattutaaautuuutyggygutgutuaguuaautyggygatuauttyggutuygutgagutgauaaaggatuaygttuuttygauuauuauagtyguutyguaguygatggtatttgyggyguuttuuttutuuuautggaaauuaaatuutgggagygtututtutgygguaguagtggayguyguygaggagggauuygagguyggauuygagguyggutuuaggaautgaguuuaagguygyggtagygatuyg

SEQ ID NO: 5 - DNA, 22 bp, Artificial Sequence
  Primer
atgaagtttatygaaggtgttt

SEQ ID NO: 6 - DNA, 24 bp, Artificial Sequence
  Primer
tttctctccaaacctcaacacctc

SEQ ID NO: 7 - DNA, 24 bp, Artificial Sequence
  Primer
gaggtgttgaggtttggagagaaa

SEQ ID NO: 8 - DNA, 24 bp, Artificial Sequence
  Primer
aacctccaccttctcatctccaca

SEQ ID NO: 9 - DNA, 24 bp, Artificial Sequence
  Primer
tgtggagatgagaaggtggaggtt

SEQ ID NO: 10 - DNA, 22 bp, Artificial Sequence
  Primer
atactactaaaccccrtcacct

SEQ ID NO: 11 - DNA, 22 bp, Artificial Sequence
  Primer
atygagaagggggtggaggtga

SEQ ID NO: 12 - DNA, 23 bp, Artificial Sequence
  Primer
aactaaaaccaaactaaataaac

SEQ ID NO: 13 - DNA, 23 bp, Artificial Sequence
  Primer
gtttatttagtttggttttagtt

SEQ ID NO: 14 - DNA, 23 bp, Artificial Sequence
  Primer
taataacctttataccacactac

SEQ ID NO: 15 - DNA, 25 bp, Artificial Sequence
  Primer
gaggygattgtggtggtygaaggaa

SEQ ID NO: 16 - DNA, 29 bp, Artificial Sequence
  Primer
aaactccctcracrctactcaaccaactc

SEQ ID NO: 17 - DNA, 28 bp, Artificial Sequence
  Primer
gaaggaaygtgattttttgttagtttag

SEQ ID NO: 18 - DNA, 40 bp, Artificial Sequence
  Primer
yggagtygaagtgatygtygagttggttgagtagygtyga